(19) United States
(12) Patent Application Publication
(10) Pub. No.: US 2024/0009461 A1
(43) Pub. Date: Jan. 11, 2024

(54) SYSTEMS AND METHODS FOR MEASURING ELECTRIC FIELD IN BIOLOGICAL TISSUES

(71) Applicant: Dignity Health, San Francisco, CA (US)

(72) Inventor: Benjamin Hendricks, San Francisco, CA (US)

(21) Appl. No.: 18/251,580

(22) PCT Filed: Nov. 1, 2021
(86) PCT No.: PCT/US2021/057572

§ 371 Date: May 3, 2023

Related U.S. Application Data
(60) Provisional application No. 63/128,223, filed on Dec. 21, 2020.
(60) Provisional application No. 63/108,992, filed on Nov. 3, 2020.

(51) Int. Cl. A61N 1/36; A61N 1/05

(52) U.S. Cl. CPC A61N 1/36139; A61N 1/0534

(57) ABSTRACT

Various embodiments of systems and methods for implantable electrode recording of an alternating electric field are disclosed herein. In particular, the system enables interradial distance-based recording of electric field differential resultant from an applied waveform between various locations within an organic structure. The determination of electric field differential between measuring contacts can enable a practitioner to create a mapping of electric field differential throughout an organic structure that can aid in understanding of how structural and material variability throughout the bodily structure affects electric field propagation through the structure.

### FIELD

The present disclosure generally relates to implantable electrode recording, and in particular to systems and methods for implantable electrode recording of an alternating electric field strength.

### BACKGROUND

Electric field, as originating from a point charge, will radially project to a correspondent point of opposite charge. When applied to a living system, calculation of the magnitude of electric field strength and the vectoral direction of field is challenging. A multitude of proposed designs and patents exist for electric field detection, but none have been designed (or optimized) for minimizing the impact on organic tissue.

It is with these observations in mind, among others, that various aspects of the present disclosure were conceived and developed.

### BRIEF DESCRIPTION OF THE DRAWINGS

FIG. 1 is a simplified schematic diagram showing a system for measuring electric field differential in an organic structure;

FIG. 2 is a simplified diagram showing a measuring contact configuration for measuring an electric field differential between a first measuring contact and a second measuring contact of the system of FIG. 1;

FIG. 3 is a simplified diagram demonstrating the electric field differential according to aspects of the present system and method in which E_{0,1} represents an electric field magnitude between a stimulating electrode and a first measuring electrode, E_{0,2} represents an electric field differential between the stimulating electrode and a second measuring electrode, and E_{1,2} represents an electric field differential between the first and second measuring electrodes;

FIGS. 4A and 4B are simplified schematics showing various configurations of a multi-contact electrode of the system of FIG. 1;

FIG. 5 is an illustration demonstrating a multi-electrode configuration of the system of FIG. 1 for determining the electric field differential between contacts on 3 electrodes;

FIG. 6 is a simplified schematic diagram showing a configuration of the system of FIG. 1 including a plurality of measuring contacts to generate a mapping of various electric field differentials across a structure;

FIG. 7 is a process flow showing a method for measuring electric field differential in an organic structure using the system of FIG. 1;

FIG. 8 is a process flow showing a sub-method for measuring an intermediate radial distance value according to the process flow of FIG. 7;

FIG. 9 is a graphical representation showing laboratory data acquired for a single depth electrode recording when a 5V input voltage is applied with a single electrode to a formalin fixed cerebral specimen at a 1 kHZ alternating electric field in which contact 4 was placed closest to the stimulating electrode and contact 1 was placed farthest form the stimulating electrode;

FIG. 10 is a graphical representation showing laboratory data acquired for a single grid electrode recording when a 5V input voltage is applied with a single electrode to a formalin fixed cerebral specimen at a 1 kHz alternating frequency in which contact 4 is placed closest to the stimulating electrode and lead 1 is placed farthest from the stimulating electrode;

FIG. 11 is a graphical representation showing laboratory data acquired for a multi-depth electrode recording when a 5V input voltage is applied with a single lead to cerebral tissue at a 1 kHZ alternating frequency;

FIG. 12 is a simplified diagram showing a computing device configured for use within the system of FIG. 1; and

FIG. 13 is a photograph showing a testing setup including the system of FIG. 1 with cerebral tissue to acquire the graphical representations of FIGS. 9-11.

Corresponding reference characters indicate corresponding elements among the view of the drawings. The headings used in the figures do not limit the scope of the claims.

### DETAILED DESCRIPTION

Various embodiments of a system and associated methods for measuring electric field within the body are disclosed herein. In particular, the system enables a practitioner to measure an electric field differential between various points within the brain, however in some embodiments the system can extend to measuring electric field differential within various points within the body. In some embodiments, the system includes a plurality of measuring contacts operable for placement at different points within the bodily structure to measure a voltage of the tissue and can further include at least one stimulating electrode operable to apply an applied voltage to the tissue. Further, the system includes a stimulating contact in communication with a computing system that enables determination of an electric field differential between the plurality of measuring contacts. In some embodiments, the system enables a practitioner to measure electric field differential through a bodily structure at various points by characterizing the electric field not only between the stimulating contact and the measuring contact, but also between measuring contacts by application of an electric field differential assessment using values measured by the measuring contacts. The determination of electric field differential between measuring contacts can enable a practitioner to create a mapping of electric field differential throughout an organic structure that can aid in understanding of how structural and material variability throughout the bodily structure affects electric field propagation through the structure. The methodology described for measurement relative to a single stimulating electrode can also be generalized to multiple stimulating electrodes.

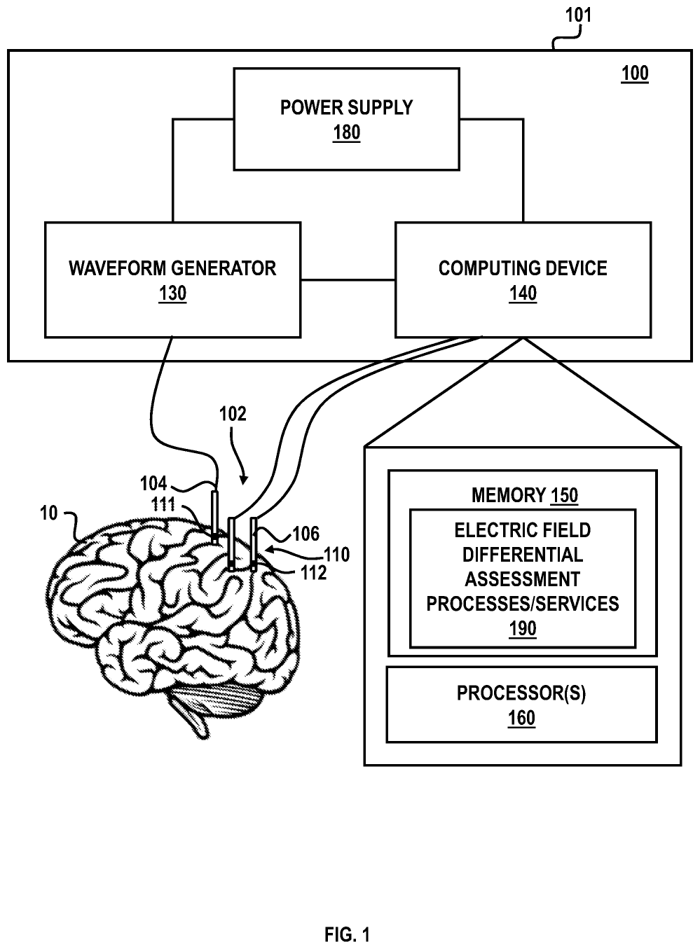

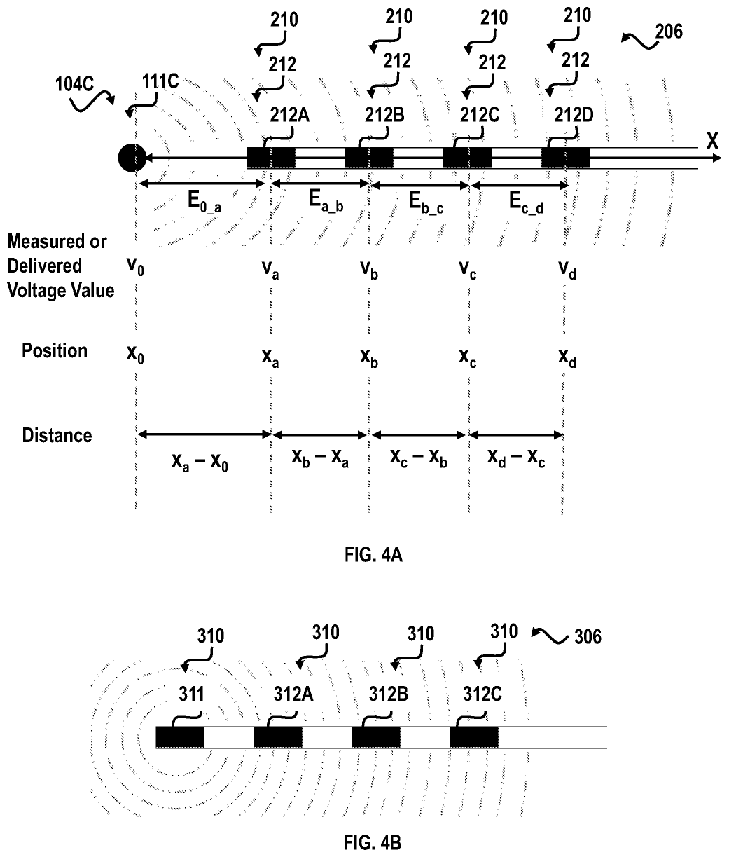

Referring to FIGS. 1-3, an electric field measurement system 100, hereinafter “system” 100, includes a plurality of electrical contacts 110 that serve as reference for voltage measurement at set locations within a Cartesian space. In some embodiments, the system 100 includes a plurality of electrical contacts 110 on at least one implantable electrode 102 such as an implantable deep brain stimulation (DBS) electrode. In some embodiments, at least one implantable electrode 102 can include a combination of single-contact electrodes and multi-contact electrodes such as a multi-contact electrode 206 or 306 (FIGS. 4A and 4B) that includes a plurality of contacts 110 configured individually for applying an applied voltage or for measuring a voltage of tissue at a point-of-contact. In some embodiments, the multi-contact electrode 206 or 306 includes variable intra-contact and inter-contact distances. At least one implantable electrode 102 can be a measuring electrode 106 that includes at least one measuring contact 112 of the plurality of electrical contacts 110 for measuring a voltage of tissue at the point-of-contact of the measuring contact 112. Further, in some embodiments, the system 100 can include a stimulating electrode 104 defining a stimulating contact 111 of the plurality of electrical contacts 110 that applies a voltage to the tissue at the point-of-contact of the stimulating contact 111. It should be noted that in some embodiments, the implantable electrode 102 can be configured to switch between stimulating and measuring roles, thereby becoming a measuring electrode 106 or a stimulating electrode 104 depending on the specific application. In some embodiments of the system 100, an electric field differential within tissue can be determined using at least two measuring contacts 112 defined along one or more measuring electrodes 106 (as each individual measuring electrode 106 can include one or more measuring contacts 112) without reference to a voltage of a stimulating electrode 104, assuming the distance from the stimulation source is known for each contact.

The physics of electric potential (voltage V) and electric field (E) permit the association of voltage with electric field within a simplified one-dimensional system or within higher dimensional systems, dependent upon the model system and desired outcome. E is representative of electric field, r is representative of a radial distance between a first point of voltage measurement and a second point of voltage measurement, V is representative of voltage at a point of measurement, and ΔV is representative of a difference in electric potential between the first and second points of voltage measurement. The simplified one-dimensional system can be approximated by the following scalar derivation −∫E·dr=ΔV. In one-dimensional space this can be simplified to: E=ΔV/r. To more accurately inform real-world estimations, this association between voltage and electric field can be represented by a vectoral quantity that reflects both a multidimensional magnitude and direction by the following derivation: −∫E·dr=ΔV. Rearranging this to solve for E permits: E=−d/dr−V\hat{r}. Given r will have a multi-dimensional component, the unit vector notation will be used for an example wherein the x, y, and z directions will be annotated with î, ĵ, and \hat{k} respectively. Given this calculation will require the use of partial derivatives the operator del (∇) can be substituted for the sum of the partial derivative in three-unit vector directions, as follows:

E=−∇V=−(î−∂V/∂x)−(j·∂V/∂y)−(\hat{k}·∂V/∂x)].

Given the complexity in describing the measurement concepts within this disclosure alone, the examples will demonstrate a system 100 for conducting electrode recordings and predictions using the one-dimensional scalar derivation for the association between voltage and electric field. However, the principles can be applied to multidimensional systems for more advanced computational predictions including vectoral direction.

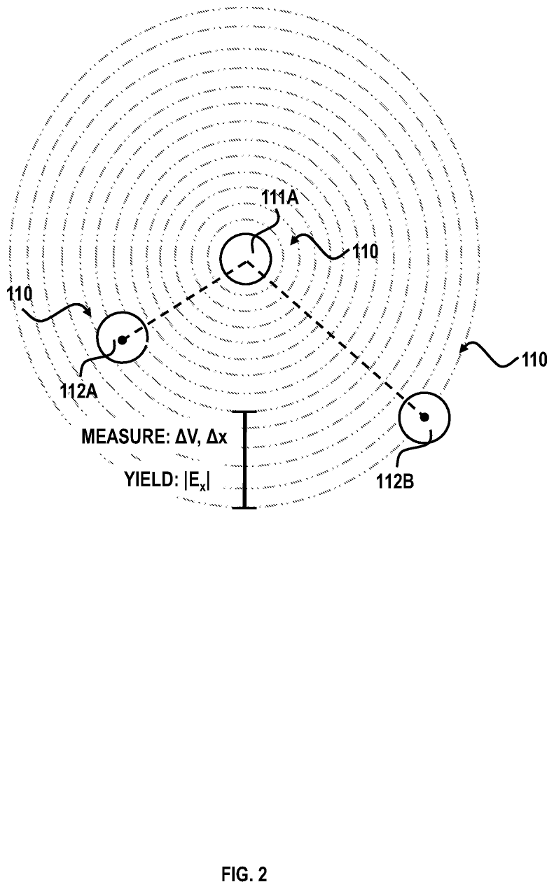

Referring to FIGS. 2-6, with knowledge of a position of each measuring contact 112 relative to the stimulating contact 111 of the plurality of contacts 110 within a Cartesian space and with voltage sampling at each measuring contact 112, the system 100 determines a magnitude |E| of an electric field differential E (i.e. a difference in electric field strength between two points) that exists between the measuring contacts 112. This can be accomplished with Eq. 1 below:

|E|=|−(ΔV)/(Δd)| (Eq. 1)

where |E| is the magnitude of electric field differential E between measuring contacts 112, ΔV is a measured voltage difference between the measuring contacts 112, and Δd is a difference in position (distance) between measuring contacts 112. Notably, in some embodiments, ΔV can be expressed as a peak voltage (Vₚₑₐₖ) or the root-mean-square (rms) voltage of a sinusoidal source such that Vᵣₘₛ=Vₚₑₐₖ/√2 or 0.707Vₚₑₐₖ.

Referring directly to FIG. 2, electric field differential measurement within a biological system defined along an X axis can be simplified to Equation 2:

|Eₓ|=|−(ΔV/Δx)| (Eq. 2)

where Eₓ is a magnitude of the electric field differential between a first measuring contact 112A and a second measuring contact 112B where the first measuring contact 112A, the second measuring contact 110B, and a first stimulating contact 111A of a first stimulating electrode 104A on an X axis. Eq. 2 states that the magnitude of the electric field difference within the X axis of a Cartesian coordinate system is equal to the absolute value of the difference in voltage between the first and second measuring contacts 110A and 110B divided by a difference in position along the X axis. Therefore, a difference in voltage (ΔV) over a difference in position (Δx) (distance) represents a directional component of electric field vector within the reference plane (e.g. the X plane). This representation assumes a homogenous decline in electric field strength over the distance (x), which can be better informed by adding more measuring contacts 112 across a substrate of interest given an isotropic substance within the X plane is not anticipated in organic tissues.

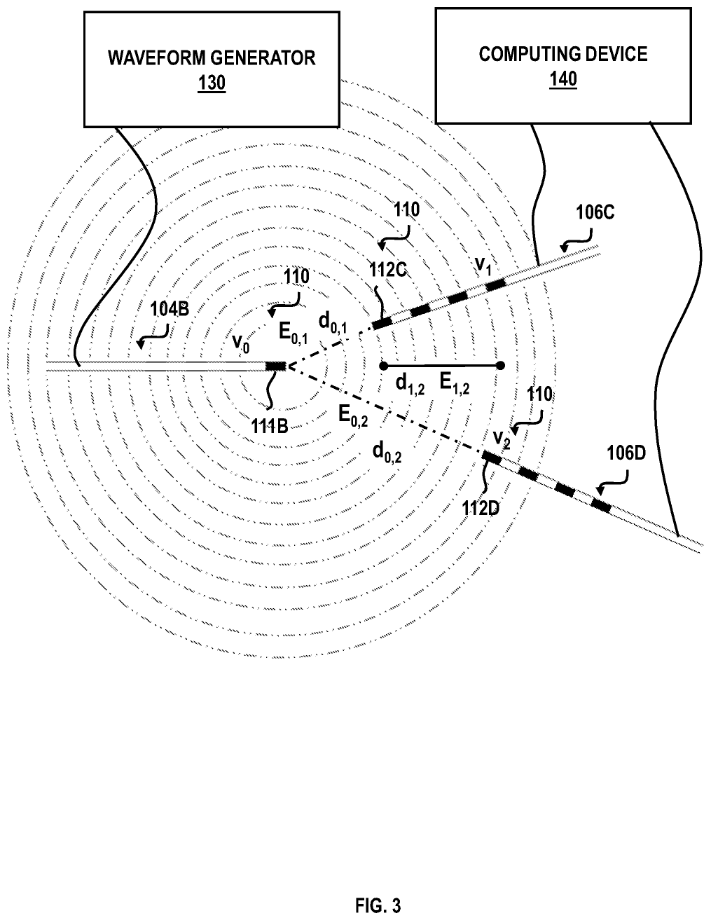

The assessment of electric field differential between various points in an organic structure can provide insights into topology of the tissue as well as assess efficacy of treatments which rely on electrical stimulation of tissue. Referring to FIGS. 1 and 3, the system 100 includes a waveform generator 130 in communication with a computing device 140 and electrically coupled with a power supply 180. The waveform generator 130 is operable for generating an applied voltage waveform that is applied to an organic structure by an associated stimulating contact 111 of a stimulating electrode 104 to generate an electric field within the organic structure. The computing device 140 is operable to sample a voltage value within an organic structure at one or more associated measuring contacts 112 of one or more measuring electrodes 106. The computing device 140 is further operable to determine a magnitude of an electric field differential E between measuring contacts 112 based on sampled voltage values at each measuring contact 112 and known distances of each measuring contact 112 relative to one another.

In some embodiments, measuring contacts 112 are in electrical communication with a voltmeter (not shown); in some embodiments the voltmeter is located within a housing 101 of the system 100 and is configured to measure a voltage at each measuring contact 112 and provide the voltage measurement to the computing device 140. The computing device 140 includes one or more processors 160 associated with a memory 150, and the memory 150 includes instructions for execution of applications including electric field magnitude assessment processes/services 190. In one example, the system 100 receives a measurement value for a particular field strength being generated. In the event that field strength is 0.7 V/cm for example and the system 100 is required to deliver 1 V/cm, the system 100 can automatically enhance the input voltage to enhance the field strength until it returns the value of 1 V/cm

FIG. 3 illustrates another configuration of the system 100 including a second stimulating electrode 104B, a third measuring electrode 106C and a fourth measuring electrode 106D. The second stimulating electrode 104B includes a second stimulating contact 111B that applies an applied voltage V₀ directly to an organic structure. The third and fourth measuring electrodes 106C and 106D each include respective third and fourth measuring contacts 112C and 112D operable to measure respective voltages V₁ and V₂ of the organic structure at point-of-contact of the respective third or fourth measuring contact 112C and 110D. A difference in intermediate radial distance relative to the second stimulating electrode contact 111B, d_{1,2}, between the third measuring contact 112C and the fourth measuring contact 112D is measured, previously known or otherwise provided. As further illustrated, in some embodiments, a distance d_{0,1} between the second stimulating contact 111B and the third measuring contact 112C and a distance d_{0,2} between the second stimulating contact 111B and the fourth measuring contact 112D can be measured, previously known or otherwise provided. In some embodiments, various imaging or locating methods such as CT or MRI scans can be utilized to quantify the positions of each contact 110 to obtain distances d_{0,1}, d_{0,2} and d_{1,2}. In some embodiments, the system 100 obtains the intermediate radial distance d_{1,2} by subtracting d_{0,1} from d_{0,2} or vice versa and in some embodiments, taking the absolute value of the result.

With further reference to the example of FIG. 3, the system 100 is operable to determine a magnitude E_{1,2} of an electric field differential between third and fourth measuring contacts 112C and 112D of the third measuring electrode 106C and the fourth measuring electrode 106D based on measured respective voltages at the measuring contact 112C of the third measuring electrode 106C, and the measuring contact 112D of the fourth measuring electrode 106D. The difference in radial distance of the third measuring contact 112C and the fourth measuring contact 112D from the second stimulating contact 111B is referred to as the intermediate radial distance value d_{1,2}. The system 100 determines the magnitude of the electric field E_{1,2} between the measuring contact 112C of the third measuring electrode 106C and the measuring contact 112D of the fourth measuring electrode 106D as:

|E_{1,2}|=|−(V₂−V₁)/d_{1,2}|. (Eq. 3)

Further, in some embodiments, the system 100 is operable to determine a magnitude E_{0,1} of an electric field differential between the second stimulating contact 111B and third measuring contact 112C based on delivered voltage at the stimulating contact 111B and the measured voltage at the third measuring contact 112C of the third electrode 106C. The system 100 determines the magnitude of the electric field differential E_{0,1} between the second stimulating contact 111B and the third measuring contact 112C of the third measuring electrode 106C as:

|E_{0,1}|−(V₁−V₀)/d_{0,1}| (Eq. 4)

Similarly, the system 100 is operable to determine a magnitude E_{0,2} of an electric field differential between the second stimulating contact 111B and the fourth measuring contact 112D of the fourth measuring electrode 106D, based on measured respective voltage values at the second stimulating contact 111B and the fourth measuring contact 112D. The system 100 determines the magnitude of the electric field differential E_{0,2} between the second stimulating contact 111B and the measuring contact 112D of the fourth measuring electrode 106D, as:

|E_{0,2}|=|−(V₂−V₀)/d_{0,2}| (Eq. 4)

In some embodiments as shown in FIG. 4A, this concept can be applied to a living biological system (for example, the brain) through the implantation of depth or grid multi-contact electrodes that each include a plurality of contacts 210 separated by known distances. A multi-contact measuring electrode 206 is illustrated defining a plurality of measuring contacts 212A-212D, where a direction of elongation of the measuring electrode 206 is considered as the X axis. In some embodiments, measuring electrode 206 includes a first measuring contact 212A as well as second, third and fourth measuring contacts 212B, 212C and 212D located at various respective positions xₐ, x_{b}, x_{c}, and x_{d} along the length of the measuring electrode 206. In the example a third stimulating contact 111C of a third stimulating electrode 104C is illustrated as delivering a voltage v₀ to a point-of-contact at position xo of the third stimulating contact 111C. It should be noted that while four measuring contacts 212A-D are illustrated, the example FIG. 4A, a multi-contact measuring electrode 206 can include any suitable quantity of measuring contacts 212. First through fourth measuring contacts 212A, 212B, 212C and 212D are operable for measuring respective voltage values vₐ, v_{b}, v_{c}, and v_{d} at the point-of-contact.

Using the relation of Eq. 2, an electric field differential can be estimated between any two measuring contacts 212A, 212B, 212C and 212D, and the knowledge of the position of the third stimulating contact 111C of the third stimulating electrode 104C. FIG. 4A in particular illustrates a magnitude E_{a_b} of electric field differential between measuring contacts 212A and 212B, a magnitude E_{b_c} of electric field differential between measuring contacts 212B and 212C, and a magnitude E_{c_d} of electric field differential between measuring contacts 212C and 212D. These values can be determined below as:

|E_{a_b}|=|−{(V_{b}−V_{a,})/[(X_{b}−X₀)−(Xₐ−X₀)]}| (Eq. 5)

|E_{b_c}|=|−{(V_{c}−V_{b,})/[(X_{c}−X₀)−(X_{b}−X₀)]}| (Eq. 6)

|E_{c_d}|=|−{(Vₐ−V_{c,})/[(Xₐ−X₀)−(X_{c}−X₀)]}| (Eq. 7)

While FIG. 4A illustrates electric field differentials between measuring contacts 212A-212D, the electric field differential determination can be applied to any combination of measuring contacts 212 from a multi-contact measuring electrode 206 or measuring contacts 112 from a single-contact measuring electrode 106. Further, referring to FIG. 4B, in some embodiments of the system 100, a multi-contact measuring electrode 306 can include a plurality of contacts 310 including a stimulating contact 311 and one or more measuring contacts 312A, 312B and 312C. Measuring electrode 306 includes a stimulating contact 311 configured to deliver a stimulating alternating current to organic tissue, and measuring contacts 312A, 312B and 312C are each configured to measure a voltage at their respective locations. In some embodiments, the intercontact distance along the measuring electrode 306 can be large (up to multiple centimeters) to avoid the requirement for a multitude of computational inputs, or extremely small (within the sub-millimetric range) to more accurately inform the dielectric properties possessed by the organic tissue housing the measuring electrode 306.

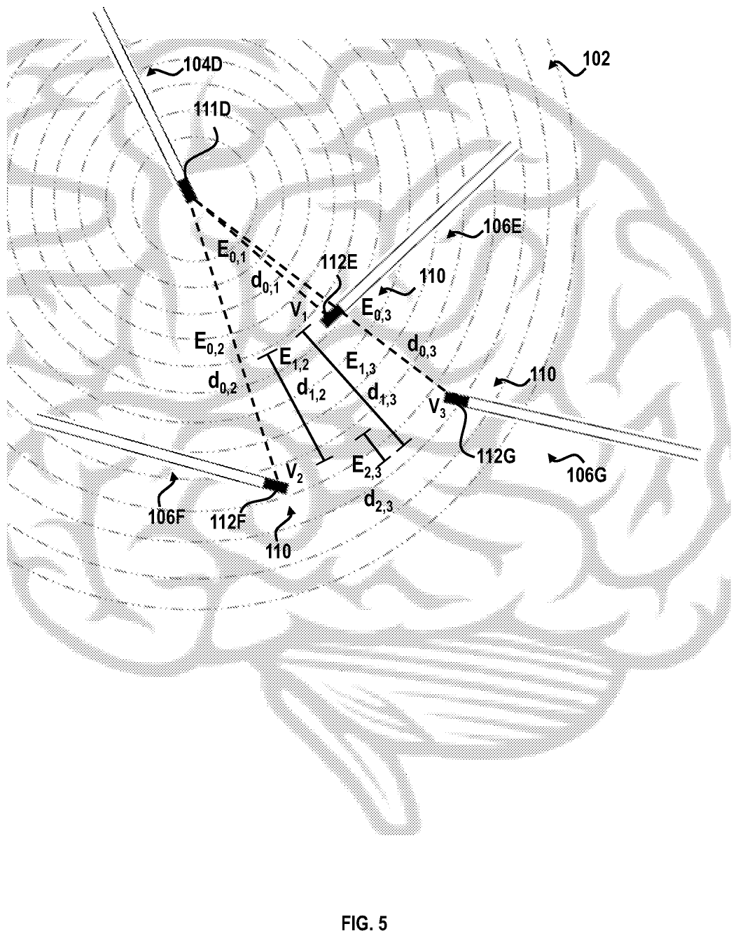

Referring to FIG. 5, measuring electrodes 106 (or 206, or 306) can be spaced out across an organic structure to measure electric field differentials between the measuring electrodes 106, such as fifth, sixth and seventh measuring electrodes 106E, 106F, and 106G. The measurement can be completed referencing the individual radial distances (d_{0,1}, d_{0,2}, d_{0,3}), from a fourth stimulating electrode 104D. In the embodiment shown, the fifth measuring electrode 106E has a fifth measuring contact 112E that measures a voltage value V₁, the sixth measuring electrode 106F has a sixth measuring contact 112F that measures a voltage value V₂, and the seventh measuring electrode 106G has a seventh measuring contact 112G that measures a voltage value V₃. As further illustrated, the fifth measuring electrode 106E and the fourth stimulating electrode 104D are separated by a distance d_{0,1}, the sixth measuring electrode 106F and the fourth stimulating electrode 104D are separated by a distance d_{0,2}, and measuring electrode 106G and the fourth stimulating electrode 104D are separated by a distance d_{0,3}. With the electric field differential assessment relation described above, the electric field differentials can be determined as:

|E_{1,2}|=|−[(V₂−V₁)/(d_{0,2}−d_{0,1})]| (Eq. 8)

|E_{1,3}|=|−[(V₃−V₁)/(d_{0,3}−d_{0,1})]| (Eq. 9)

|E_{2,3}|=|−[(V₃−V₂)/(d_{0,3}−d_{0,2})]| (Eq. 10)

This differential assessment permits analysis of traversing electric field in a region between measuring electrodes 106, and not simply between a stimulating electrode 104 and a measuring electrode 106 as in the example of FIG. 3. The electric field differential assessment between measuring contacts 112, rather than exclusively between a measuring contact 112 and a stimulating contact 111, can enable a practitioner to understand electric field propagation through an organic structure while inserting fewer measuring contacts 112 into the tissue.

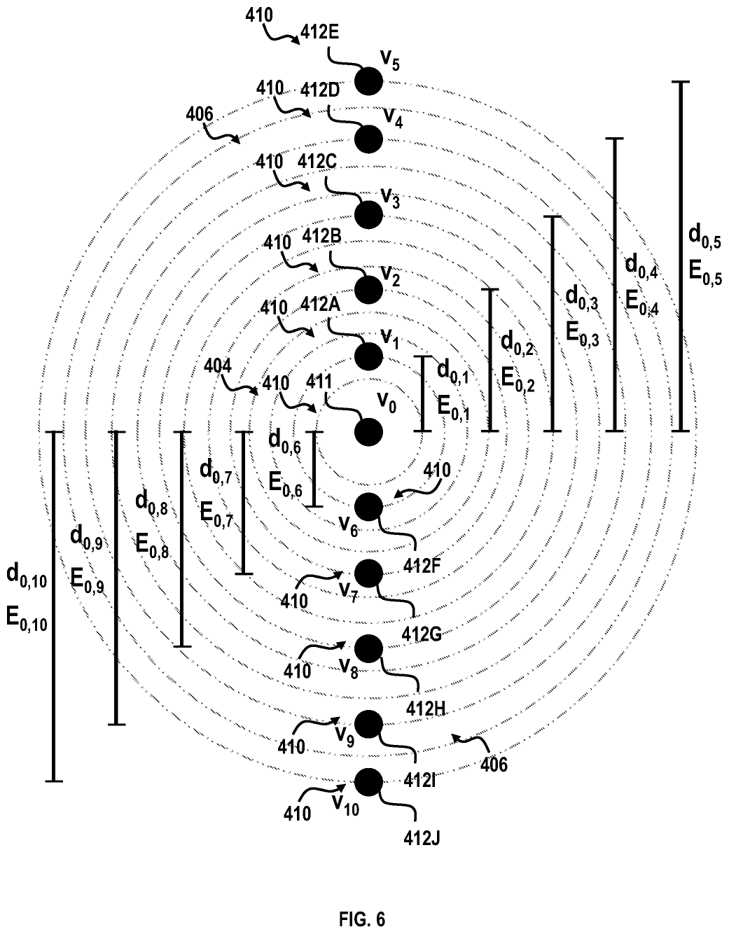

Referring to FIG. 6, the system 100 can be scaled to assess electric field differentials across a plurality of locations on an organic structure. In particular, FIG. 6 illustrates a hypothetical measuring electrode and stimulating electrode configuration of the system 100 that includes a plurality of contacts 410 including ten measuring contacts 412 (412A-412J) and one stimulating contact 411, however other configurations of measuring contacts 412 (or 112, 212 or 312) and stimulating contacts 411 (or 111, or 311) are contemplated. While it is shown that the electric field differential assessment at different measuring contacts 412 can be performed with respect to the stimulating contact 411, electric field differential assessment made directly between different measuring contacts 412 can also be performed as described above. In some embodiments, some contacts 410 of the plurality of contacts 410 are operable for switching between functionalities as a stimulating contact 411 or a measuring contact 412. In some embodiments, electric field differentials between a plurality of contacts 410 within tissue can be used to create mappings showing electric field propagation variation through tissue between measuring contacts 410. This will permit correction and adjustment of predictions made using this system according to the dielectric properties (conductivity and permittivity) of the organic tissue that are relevant to a particular region of tissue.

It should be noted that the use of determining electric field differential between measuring contacts 112/212/312/412, rather than exclusively between a measuring contact 112/212/312/412 and a stimulating contact 111/311/411, can enable a practitioner to understand electric field propagation through organic tissue while inserting fewer measuring contacts 112/212/312/412 into the tissue. In particular, in some embodiments, stimulating contacts 111 are not necessary to understand the field propagation. That is realized based on the recordings of the measuring electrodes 106. It is feasible to extrapolate the dispersion of electric field across the tissue based on a few measuring electrode recordings and thereby predict the dispersion of field within tissue (based on radial distance) that lacks the presence of additional measuring electrodes.

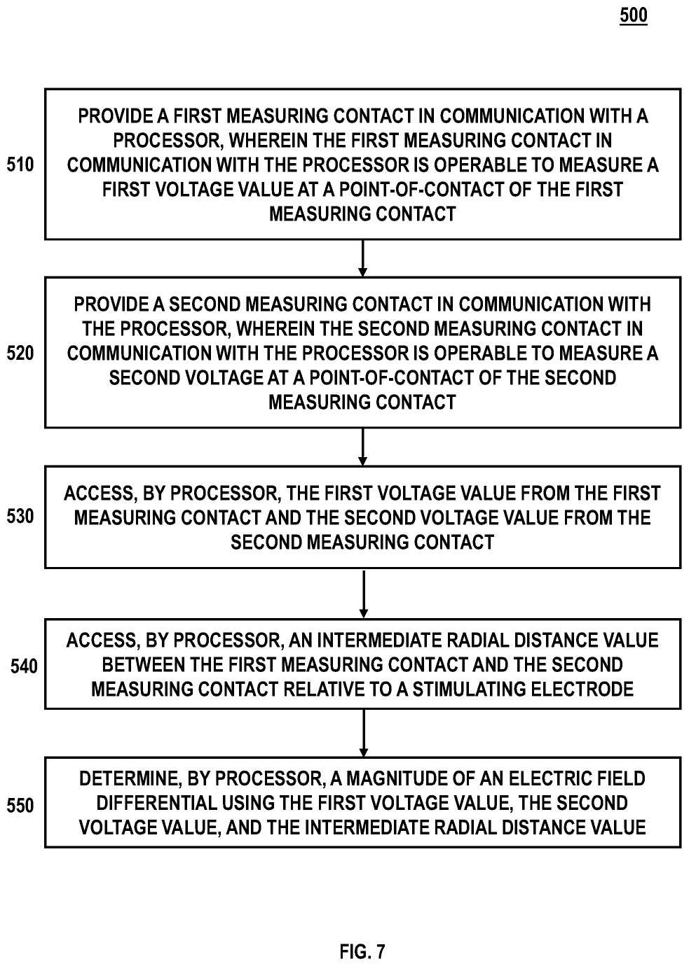

A method 500 of determining electric field differential between measuring contacts is illustrated in FIG. 7. At block 510, the system 100 provides a first measuring contact 112/212/312/412 in communication with the processor 160, wherein the first measuring contact 112/212/312/412 in communication with the processor 160 is operable to measure a first voltage value at a point-of-contact of the first measuring contact 112/212/312/412. At block 520, the system 100 provides a second measuring contact 112/212/312/412 in communication with the processor 160, wherein the second measuring contact 112/212/312/412 in communication with the processor 160 is operable to measure a second voltage value at a point-of-contact of the second measuring contact 112/212/312/412. At block 530, the system 100 accesses, by the processor 160, the first voltage value from the first measuring contact 112/212/312/412 and the second voltage value from the second measuring contact 112/212/312/412.

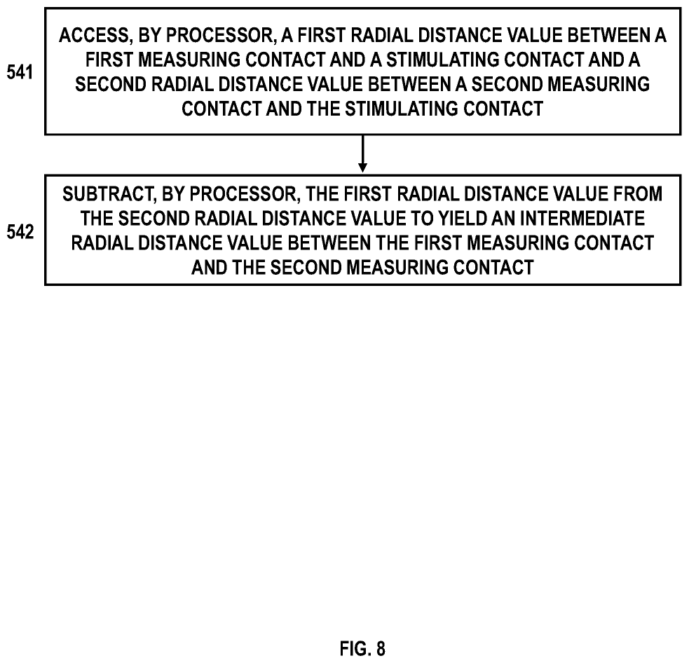

At block 540, the system 100 accesses, by the processor 160, an intermediate radial distance value between the first measuring contact 112/212/312/412 and the second measuring contact 112/212/312/412. In particular, as shown in FIG. 8, at block 541, the system 100 accesses, by the processor 160, a first radial distance value between the first measuring contact 112/212/312/412 and the stimulating contact 111/311/411 and a second radial distance value between a second measuring contact 112/212/312/412 and the stimulating contact 111/311/411. Subsequently, at block 542, the system 100 subtracts, by processor 160, the first radial distance value from the second radial distance value to yield an intermediate radial distance value between the first measuring contact 112/212/312/412 and the second measuring contact 112/212/312/412. At block 550, the system 100 determines, by the processor 160, a magnitude of an electric field using the first voltage value, the second voltage value, and the intermediate distance value according to Eq. 1 and variations on Eq. 1 described herein.

### Single Electrode Magnitude Measurements

### Depth Electrode Testing

To test this hypothesis a stimulating electrode was implanted into a cadaveric (formalin fixed) specimen with the grounding lead placed within the same specimen. A DBS depth electrode was placed horizontally into the specimen with contact 1 being placed farthest from the input voltage source, and contact 4 being closest. The measuring electrode is connected to a desktop digital data acquisition (DAQ). A single channel waveform generator is used to provide an input voltage of 5V (10 Vpp) with an alternating frequency of 1 kHz.

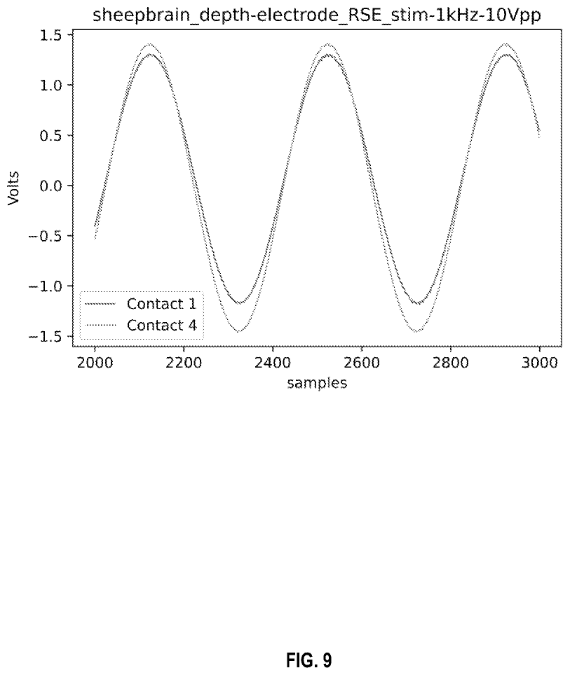

FIG. 9 shows a graphical representation of laboratory data acquired for a single depth electrode recording when a 5V input voltage (10V peak-to-peak) was applied with a single electrode to a formalin fixed cerebral specimen at a 1 kHz alternating electric field. Contact 4 was placed closest to the stimulating electrode and Contact 1 was placed farthest.

The measurements from the depth electrode permitted the following data:

Vₚₚ=2.495 V, Vₘₐₓ=1.309 V, Vₘᵢₙ=−1.186 V Contact 1:

Vₚₚ=2.866 V, Vₘₐₓ=1.408 V, Vₘᵢₙ=−1.459 V Contact 4:

In some embodiments of a depth electrode, the inter-contact distance is 0.5 mm and the intra-contact distance is 1.5 mm. Thus, the distance between the center of contact 1 and the center of contact 4 is 6 mm (0.75 mm+0.5 mm+1.5 mm+0.5 mm+1.5 mm+0.5 mm+0.75 mm) or 0.6 cm. For this comparison the electrodes will be considered in a linear 1-dimensional plane (X), along which axis the stimulating electrode has been placed. This calculation without disclosure of the stimulating source location (X₀) is permissible because the stimulating source was within the 1D radial plane of X and therefore (X₄−X₀)−(X₁−X₀)→X₄−X₀−X₁+X₀→X₄−X₁. The zero point along X will be arbitrarily assigned to the center of Contact 4. Therefore, the electric field peak magnitude will be represented by |Eₓ|=|(ΔV/Δx)|=|(V₄ₚₑₐₖ−V₁ₚₑₐₖ)/(X_{4,0}−X_{1,0})|=|(1.408−1.309)/(0−0.6)|=0.165V/cm.

To calculate the peak electric field differential magnitude estimate between contact 4 and the stimulating electrode, the same formula can be applied with the knowledge that contact 4 was 9.1 mm from the stimulating electrode, which was stimulating with an amplitude Vmax=5V (or 10 Vpp). Therefore: |Eₓ|=|(ΔV)/(Δd)|=|(5−1.408)/0.91|=3.991V/cm. If it was desired to present this value as RMS alternating electric field magnitude (E_{RMS}) the value could simply be multiplied by calculated using Eₚₑₐₖ/√2 or 0.707Eₚₑₐₖ. Therefore, based on the above calculation evidence is obtained that within the 1-dimensional plane (x) the peak electric field magnitude between the stimulating electrode and the measuring electrode can be estimated as 3.991 V/cm. This insight provides a more accurate understanding of the change in electric field strength within the tissue than simply comparing the calculating the electric field strength at each individual contact relative to the stimulating electrode. This inter-contact calculation of electric field magnitude can also serve to educate predictive analytics of a tissue housing the measuring electrode(s) contacts to understand how certain pathological conditions, such as brain swelling, might impact traversing electric field.

### Grid Electrode Testing

A similar analysis was conducted using a grid electrode placed along the surface of the brain. This electrode has 4 contacts arranged horizontally along the surface of the specimen with contact 4 being placed farthest from the input voltage source and contact 4 being closest. The measuring electrode is connected to a desktop digital data acquisition (DAQ). A single channel waveform generator is used to provide an input voltage amplitude of 5V (10 Vpp) with an alternating frequency of 1 kHz.

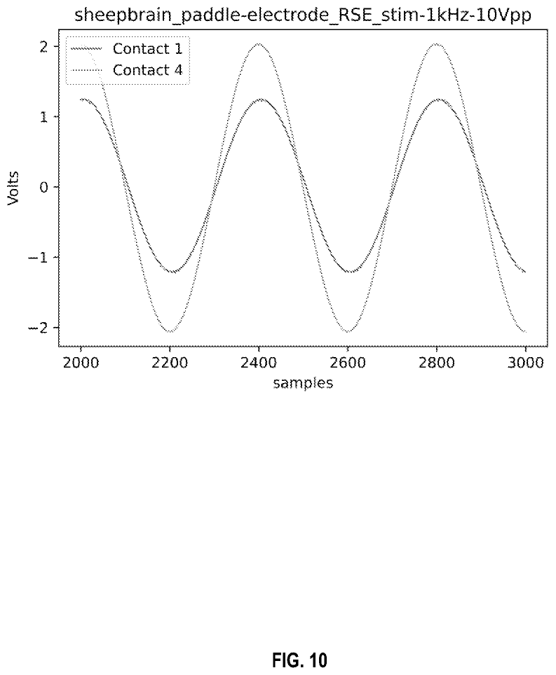

FIG. 10 shows a graphical representation of laboratory data acquired for a single grid electrode recording when a 5V input voltage was applied with a single electrode to a formalin fixed cerebral specimen at a 1 kHz alternating electric field. Contact 4 was placed closest to the stimulating electrode and Contact 1 was placed farthest away. The stimulation source was placed such that it was just below the cerebral surface.

The measurements from the grid electrode permitted the following data:

Vpp=2.475 V, Vmax=1.252 V, Vmin=−1.224 V Contact 1:

Vpp=4.099 V, Vmax=2.034 V, Vmin=−2.065 V Contact 4:

In some embodiments, an intercontact distance is 6.2 mm, and the intracontact distance is 4 mm. This makes the distance between the center of contact 1 and the center of contact 4 to be 30.6 mm (2.0 mm+6.2 mm+4.0 mm+6.2 mm+4.0 mm+6.2 mm+2.0 mm) or 3.06 cm. For this example the electrodes will be considered in a linear 1-dimensional plane (X), along which axis the stimulating electrode has been placed. This calculation without disclosure of the stimulating source location (X₀) is permissible because the stimulating source was within the 1D radial plane of X and therefore (X₄−X₀)−(X₁−X₀)→X₄−X₀−X₁+X₀→X₄−X₁. The zero point along X will represent the center of Contact 4. Therefore, the electric field magnitude will be represented by |Eₓ|=|−(ΔV/AX)=|−(V₄ₚₑₐₖ−V₁ₚₑₐₖ)/(X_{4,0}−X_{1,0})|=(2.034−1.252)/(0−3.06)=0.255 V/cm.

A similar calculation of the peak electric field magnitude estimate between contact 4 and the stimulating electrode can be conducted with the knowledge that contact 4 was 9.0 mm from the stimulating electrode, which was stimulating with a Vmax=5V (or 10 Vpp). Therefore: |Eₓ|=|−(ΔV)/(Δd)|=|−(5−2.034)/0.91=3.300V/cm.

### Multi-Electrode Magnitude Measurement

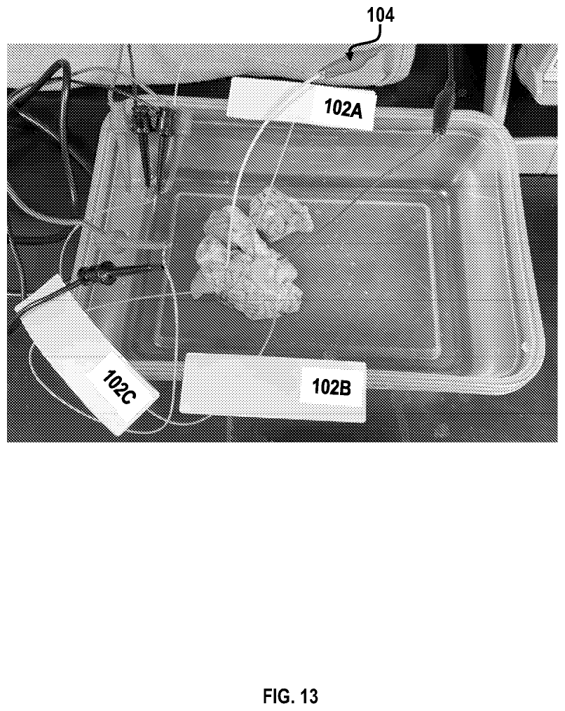

Multi-electrode measurement configurations were demonstrated in the lab where fresh Ovis aries cerebral tissue was placed in a dish. Notably, there will be innate error in this estimation of radially dispersed alternating electric field magnitude (Eₓ) due to lack of isotropic tissue (i.e. differences in tissue conductivity and permittivity), assumption of a uniform electric field, and assumption of a singular plane of reference the radial distance will be represented on, X. One stimulating electrode 104 was placed along the margin of the cerebral tissue (white lead). Three measuring electrodes 102A, 102B and 102C were placed in a triangular configuration, as demonstrated in FIG. 13. The distance (d_{0,1}) between electrodes 102A and 104 is 1.21 cm. The distance (d_{0,2}) between electrodes 102B and 104 is 2.65 cm. The distance (d_{0,3}) between electrodes 102C and 104 is 1.43 cm.

A single channel waveform generator is used to provide an input voltage of 5V with an alternating frequency of 1 kHz via the stimulating electrode. The measuring electrodes were connected to a desktop digital data acquisition (DAQ).

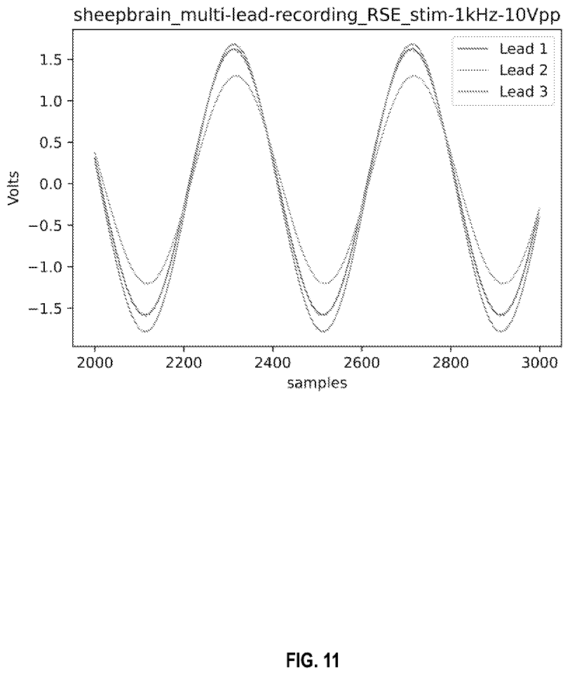

FIG. 11 shows a graphical representation of laboratory data acquired for a multi-depth electrode recording when a 5V input voltage (10 Vpp) was applied with a single electrode to Ovis aries cerebral tissue at a 1 kHz alternating electric field.

The measurements from the depth electrodes permitted the following data:

Vₚₚ=3.227 V, Vₘₐₓ=1.628 V, Vₘᵢₙ=−1.599 V Lead 1:

Vₚₚ=2.512 V, Vₘₐₓ=1.302 V, Vₘᵢₙ=−1.210 V Lead 2:

Vₚₚ=3.474 V, Vₘₐₓ=1.685 V, Vₘᵢₙ=−1.789 V Lead 3:

|E_{1,2}|=|−(ΔV/Δd)|=|−(V₂−V₁)/(d_{2,0}−d_{1,0})|=|−(1.302−1.628)/(2.65−1.21)|=0.226 V/cm

|E_{2,3}|=|−(ΔV/Δd)|=|−(V₃−V₂)/(d_{3,0}−d_{2,0})|=|−(1.685−1.302)/(1.43−2.65)|=0.314 V/cm

|E_{1,3}|=|−(ΔV/Δd)|=|−(V₃−V₁)/(d_{3,0}−d_{1,0})|=|−(1.685−1.628)/(1.43−1.21)|=0.259 V/cm

The result of these calculations demonstrates the expected results of peak electric field differential simplified to be projected along a single radial dimension from the stimulating electrode 104, based on the distance from the voltage source to the electrodes of interest. The same calculations can be conducted between the input voltage source and the individual electrode contacts to provide an estimate of the electric field magnitude between the measuring contact 102A/102B/102C and the stimulating electrode 104 (not shown due to redundancy with above examples).

The idea presented within this disclosure will allow for correction of the assumption that uniform electric field is maintained between a stimulation source and a single measuring electrode contact. When multiple electrode contacts are referenced between the source (for stimulating strength) and other measuring electrodes (for modification) the electric field dispersion can be estimated in the intervening region. Notably, these examples do not include multiple stimulating electrodes or examples with multiple stimulating electrodes that demonstrate phase shifting of the waveforms for stimulation within multi-electrode stimulation configurations. If multiple stimulating electrodes are present than the waveform of stimulation would need to be referenced by the computing device 140 to isolate the stimulating electrode exemplifying peak voltage at the exact moment in time that the measuring electrode is sampling the tissue. In that situation the stimulating electrode currently demonstrating the highest voltage would be the source for electric field stimulation to the measuring electrode. Phase shifting between stimulating electrodes is an advantageous method for maximizing the electric field magnitude within organic tissue and given there will be an offset between the sinusoidal stimulating waves for example, the computing device 140 will be able to isolate the stimulating electrode providing the momentary peak in voltage and thereby permit computation of the electric field magnitude.

The methodology described within this disclosure utilizing depth or grid electrodes with multiple contacts permit single-electrode recordings of electric field magnitude within a single dimension along the axis of the electrode. Application of the advanced unit vector-based mathematics described within the introduction would permit multi-dimensional calculations of this metric (not shown). It was also demonstrated that electric field magnitude measurements between electrodes can be computed based on contact measurements from separate electrodes. Lastly, it demonstrated the feasibility and methodology for measuring electric fields within tissue by comparing the applied voltage with point measurements taken within the tissue. This approach does oversimplify the calculation based on the assumption of a uniform electric field within the substance being implanted but provides a useful approximation of electric field magnitude. This can also assist with the planning and execution of accommodation for anatomical obstacles when strategic cerebral implantation is necessary. The ability to measure electric field magnitude described in this disclosure permits real-time feedback wherein the stimulating electrode(s) are contained in a closed loop system, to achieve a desired electric field magnitude at a target tissue region.

### Computer-Implemented System

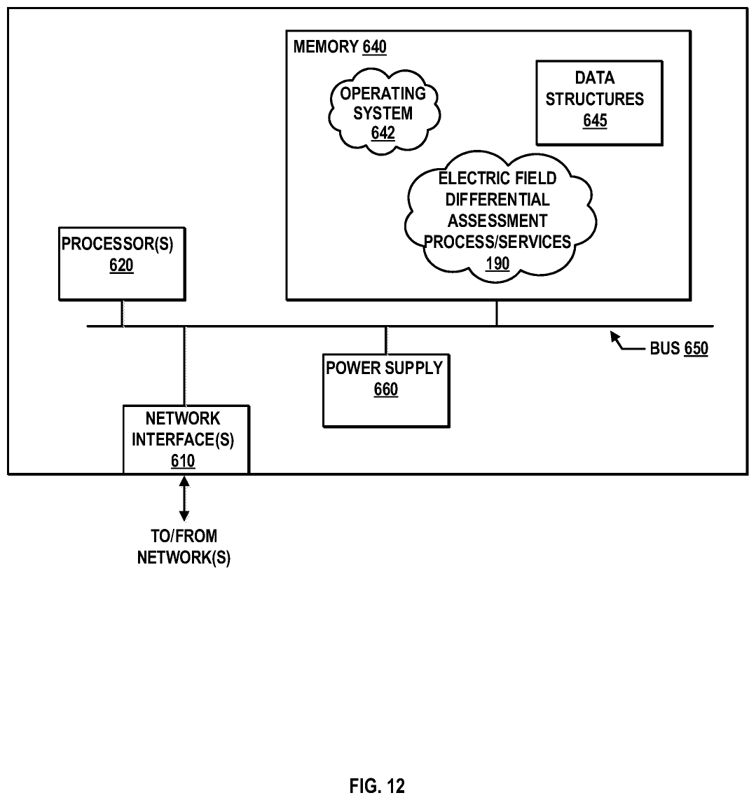

FIG. 12 is a schematic block diagram of an example device 600 that may be used with one or more embodiments described herein, e.g., as a component of system 100 and/or as computing device 140 shown in FIG. 1.

Device 600 can include one or more network interfaces 610 (e.g., wired, wireless, PLC, etc.), at least one processor 620 which in some embodiments is processor 160 of FIG. 1, and a memory 640 interconnected by a system bus 650, as well as a power supply 660 (e.g., battery, plug-in, etc.). In some embodiments, the processor 620 can be external (i.e. non-implanted) and capable of wirelessly interfacing with implanted components of the system 100.

Network interface(s) 610 include the mechanical, electrical, and signaling circuitry for communicating data over the communication links coupled to a communication network. Network interfaces 610 are configured to transmit and/or receive data using a variety of different communication protocols. As illustrated, the box representing network interfaces 610 is shown for simplicity, and it is appreciated that such interfaces may represent different types of network connections such as wireless and wired (physical) connections. Network interfaces 610 are shown separately from power supply 660, however it is appreciated that the interfaces that support PLC protocols may communicate through power supply 660 and/or may be an integral component coupled to power supply 660.

Memory 640 includes a plurality of storage locations that are addressable by processor 620 and network interfaces 610 for storing software programs and data structures associated with the embodiments described herein. In some embodiments, device 600 may have limited memory or no memory (e.g., no memory for storage other than for programs/processes operating on the device and associated caches).

Processor 620 includes hardware elements or logic adapted to execute the software programs (e.g., instructions) and manipulate data structures 645. An operating system 642, portions of which are typically resident in memory 640 and executed by the processor, functionally organizes device 600 by, inter alia, invoking operations in support of software processes and/or services executing on the device. These software processes and/or services may include electric field magnitude or direction (when applied to multi-dimensional mathematics) assessment processes/services 190 described herein. Note that while electric field assessment processes/services 190 is illustrated in centralized memory 640, alternative embodiments provide for the process to be operated within the network interfaces 610, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the electric field assessment processes/services 190 is shown as a standalone process, those skilled in the art will appreciate that this process may be executed as a routine or module within other processes.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a first measuring contact in communication with a processor, wherein the first measuring contact in communication with the processor is operable to measure a first voltage value at a point-of-contact of the first measuring contact; and
a second measuring contact in communication with the processor, wherein the second measuring contact in communication with the processor is operable to measure a second voltage value at a point-of-contact of the second measuring contact;
wherein the processor includes instructions which, when executed, cause the processor to:
access the first voltage value from the first measuring contact and the second voltage value from the second measuring contact;
access an intermediate radial distance value representative of a difference in a distance between the first measuring contact and the second measuring contact; and
determine a magnitude of an electric field using the first voltage value, the second voltage value, and the intermediate radial distance value.

2. The system of claim 1, wherein the first measuring contact is a single contact on an implantable depth electrode.

3. The system of claim 1, wherein first measuring contact is a contact of a plurality of contacts on a multi-contact electrode.

4. The system of claim 3, wherein the implantable multi-contact electrode includes at least one stimulating contact configured to apply an applied voltage at point-of-contact.

5. The system of claim 1, wherein the instructions which, when executed, further cause the processor to:
determine a first distance value representative of a difference in a distance between the first measuring contact and a stimulating contact, and a second distance value representative of a distance between the second measuring contact and the stimulating contact; and
subtract the second distance value from the first distance value to obtain the intermediate radial distance value between the first measuring contact and the second measuring contact.

6. The system of claim 5, wherein the instructions which, when executed, further cause the processor to:
quantify a first position of the first measuring contact and a second position of the second measuring contact using imaging.

7. The system of claim 1, wherein the magnitude of the electric field is determined using a single dimensional relation:

|E|=|−(V₂−V₁)/(Δd)|

wherein Δd is representative of the first distance value and wherein V₁ and V₂ are respectively representative of the first voltage value and the second voltage value.

8. The system of claim 1, wherein a magnitude and a directionality of the electric field are determined using a relation:
E=−∇V=[−(î−∂V/∂x)−(ĵ·∂V/∂y)−(\hat{k}·∂V/∂x)]
wherein ∂V is representative of a rate of change of voltage, î is representative of a unit vector notation for an x direction, ĵ is representative of a unit vector notation for a y direction, and \hat{k} is representative of a unit vector notation for a z direction.

9. The system of claim 1, further comprising a stimulating contact in communication with a waveform generator, wherein the stimulating contact in communication with the waveform generator is operable to apply an applied voltage at point-of-contact.

10. The system of claim 9, wherein the stimulating contact is in further communication with a processor, and wherein the processor includes instructions which, when executed, cause the processor to:
access an applied voltage value associated with the stimulating contact;
access a second distance value representative of a physical distance between the first measuring contact and the stimulating contact; and
determine a magnitude of an electric field using the applied voltage value, the first voltage value, and the second distance value.

11. The system of claim 1, wherein the instructions which, when executed, further cause the processor to:
generate a mapping by determining the magnitude of electric field between a plurality of measuring contacts at a plurality of locations across an organic structure.

12. The system of claim 1, wherein a measuring contact is configured to switch between a measuring contact role and a stimulating contact role.

13. A method, comprising:
providing a first measuring contact in communication with a processor, wherein the first measuring contact in communication with the processor is operable to measure a first voltage value at a point-of-contact of the first measuring contact;
providing a second measuring contact in communication with the processor, wherein the second measuring contact in communication with the processor is operable to measure a second voltage value at a point-of-contact of the second measuring contact;
accessing, by the processor, the first voltage value from the first measuring contact and the second voltage value from the second measuring contact;
accessing, by the processor, an intermediate radial distance value representative of a difference in a distance between the first measuring contact and the second measuring contact; and
determining, by the processor, a magnitude of an electric field using the first voltage value, the second voltage value, and the intermediate radial distance value.

14. The method of claim 13, further comprising:
determining a first distance value representative of a difference in a distance between the first measuring contact and a stimulating contact, and a second distance value representative of a distance between the second measuring contact and the stimulating contact; and
subtracting the second distance value from the first distance value to obtain the intermediate radial distance value between the first measuring contact and the second measuring contact.

15. The method of claim 14, further comprising:
quantifying a first position of the first measuring contact and a second position of the second measuring contact using imaging.

16. The method of claim 13, wherein the magnitude of the electric field is determined using a relation:

|E|=|−(V₂−V₁)/(Δd)|

wherein Δd is representative of the first distance value and wherein V₁ and V₂ are respectively representative of the first voltage value and the second voltage value.

17. The method of claim 13, wherein a magnitude and a directionality of the electric field are determined using a relation:
E=−∇V=[−(î·∂V/∂x)−(ĵ·∂V/∂y)−(\hat{k}·∂V/∂x)]
wherein ∂V is representative of a rate of change of voltage, î is representative of a unit vector notation for an x direction, ĵ is representative of a unit vector notation for a y direction, and \hat{k} is representative of a unit vector notation for a z direction.

18. The method of claim 13, further comprising:
applying an applied voltage at point-of-contact using a stimulating contact in communication with a waveform generator.

19. The method of claim 18, wherein the stimulating contact is in further communication with a processor, and wherein the processor includes instructions which, when executed, cause the processor to:
access an applied voltage value associated with the stimulating contact;
access a second distance value representative of a physical distance between the first measuring contact and the stimulating contact; and
determine a magnitude of an electric field using the applied voltage value, the first voltage value, and the second distance value.

20. The method of claim 13, further comprising:
generating a mapping of electric field between a plurality of measuring contacts at a plurality of locations across an organic structure.
